(19) United States
(12) Patent Application Publication
(10) Pub. No.: US 2021/0088510 A1
(43) Pub. Date: Mar. 25, 2021

(54) MODIFIED PARTICLE, METHOD FOR MANUFACTURING MODIFIED PARTICLE, AND DETECTION APPARATUS

(71) Applicant: Panasonic Intellectual Property Management Co., Ltd., Osaka (JP)

(72) Inventor: TAKASHI KANNO, Hyogo (JP)

(21) Appl. No.: 17/111,616

(22) Filed: Dec. 4, 2020

Related U.S. Application Data
(63) Continuation of application No. PCT/JP2019/041831, filed on Oct. 25, 2019.

(30) Foreign Application Priority Data
Dec. 17, 2018 (JP) 2018-235723

(51) Int. Cl. G01N 33/543; G01N 33/58; G01N 33/84

(52) U.S. Cl. CPC G01N 33/5434; G01N 33/84; G01N 33/58

(57) ABSTRACT

The present disclosure provides a modified particle in which deterioration of a specific binding substance fixed to the surface of the particle is reduced. The modified particle according to the present disclosure contains a particle, a specific binding substance that has a property of specifically binding to an analyte and that has been fixed to the surface of the particle, and an amino sugar molecule fixed to the surface of the particle with an amide bond.

### BACKGROUND

### 1. Technical Field

The present disclosure relates to a modified particle for detecting an analyte in a sample, a method for manufacturing a modified particle, and a detection apparatus.

### 2. Description of the Related Art

In recent years, biosensors exploiting a bioactive substance such as an antibody (hereafter referred to as a specific binding substance) have been used in the medical field and the biochemical field.

For example, Japanese Unexamined Patent Application Publication (Translation of PCT Application) No. 04-503404 discloses an assay kit having a configuration in which a specific binding substance for capturing an analyte is absorbed or bound to a substrate, a specific binding substance for detecting the presence or absence of the captured analyte is arranged on a substrate, and freeze-drying is performed so as to be used as the situation demands.

### SUMMARY

Incidentally, as another method in which a specific binding substance is used, there is also a method in which a granular base material is used. In this method, a granular base material to which a specific binding substance is bound in advance is bound to an analyte, and the presence or absence of the analyte and the amount of the analyte present are detected by using various detection methods in accordance with the characteristics of the granular base material bound to the analyte. In the above-described method, an extremely small change in the amount of the analyte present or the like has to be detected, and higher detection sensitivity is required in many cases.

One non-limiting and exemplary embodiment provides a modified particle and the like that can realize higher detection sensitivity in detection of an analyte.

In one general aspect, the techniques disclosed here feature a modified particle containing a particle, a specific binding substance that has a property of specifically binding to an analyte and that has been fixed to the surface of the particle, and an amino sugar molecule fixed to the surface of the particle with an amide bond.

According to the present disclosure, a modified particle and the like that can realize higher detection sensitivity can be provided.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a schematic diagram illustrating an example of a modified particle according to an embodiment;

FIGS. 2A and 2B are flow charts illustrating an example of a method for manufacturing modified particles according to an embodiment;

FIG. 3 is a schematic configuration diagram illustrating an example of a detection apparatus according to an embodiment;

FIG. 4 is a diagram illustrating modified particles when used for a detection apparatus;

FIG. 5 is a schematic diagram illustrating an example of a two-dimensional image output from a detection apparatus according to an embodiment;

FIGS. 6A and 6B are diagrams illustrating a test method for an adsorption test of a modified particle according to an example; and

FIG. 7 is a diagram illustrating the results of the adsorption test of the modified particle according to the example.

### DETAILED DESCRIPTION

### Underlying Knowledge Forming Basis of the Present Disclosure

A specific binding substance is susceptible to damage due to heat or drying. For example, part of the structure of the specific binding substance is denatured due to heat or drying and the function deteriorates. This frequently causes problems in the case in which the specific binding substance is fixed and used on the surface of a sensor substrate or a particle. Since such functional deterioration of the specific binding substance directly leads to deterioration in detection sensitivity in detection of an analyte, it is desirable that the functional deterioration be minimized.

In this regard, Japanese Unexamined Patent Application Publication (Translation of PCT Application) No. 04-503404 discloses an assay kit which has a configuration in which a specific binding substance for capturing an analyte and a specific binding substance for detecting the presence or absence of the captured analyte are arranged on a substrate, and in which freeze-drying is performed so as to be used as the situation demands. Freeze-drying enables a specific binding substance to endure long-term storage to some extent and to be immediately restored to a usable state by adding a solution at the time of use.

However, it cannot be said that the above-described effects are always achieved since the assay kit in the related art is produced by using freeze-drying, and some specific binding substances are not suitable for freeze-drying in accordance with the types of the substances. Therefore, it cannot be said that the assay kit in the related art has a sufficient effect of protecting a specific binding substance from functional deterioration. In particular, due to structural and functional deterioration of the specific binding substance (hereafter referred to as deterioration of the specific binding substance), impurities and the like in the sample may nonspecifically adsorb or bind to deteriorated portions of the specific binding substance (hereafter referred to as nonspecific adsorption). Further, when the above-described technology is applied to a method using a granular base material to which the specific binding substance is fixed, there is a problem in that aggregation of the granular base material due to nonspecific adsorption increases and that nonspecific adsorption to a structure delimiting the space in which the granular base material is stored increases.

Accordingly, the present disclosure provides a modified particle in which deterioration of a specific binding substance fixed to the surface of the particle is reduced and provides a method for manufacturing the modified particle. In addition, the present disclosure provides a detection apparatus that can detect an analyte by using the modified particle.

### Outline of Disclosure

An outline of an aspect of the present disclosure is as described below.

A modified particle according to an aspect of the present disclosure includes a particle, a specific binding substance that has a property of specifically binding to an analyte and that has been fixed to the surface of the particle, and an amino sugar molecule fixed to the surface of the particle with an amide bond.

Accordingly, the amino sugar molecule is stably fixed to the surface of the particle. The amino sugar molecule stably fixed to the surface of the particle is not separated from the surface of the particle in a liquid. In this regard, since a hydroxy group (OH group) included in the amino sugar molecule acts instead of a water molecule, the hydrophobic portion of the specific binding substance is not exposed and deterioration is reduced. Reducing deterioration of the specific binding substance on the surface of the particle also suppresses interaction between modified particles or between the modified particle and a section with which the modified particle may come into contact during detection so as to inhibit nonspecific adsorption of these. Consequently, detection sensitivity of the analyte can be enhanced.

For example, the modified particle according to the aspect of the present disclosure may include a base material and an organic film covering at least part of the surface of the base material, and the amino sugar molecule may be fixed to the organic film with an amide bond.

Accordingly, the modified particle in which the amino sugar molecule is arranged on the surface can be readily formed by selecting an organic film that can readily form an amide bond with the amino sugar molecule.

For example, the modified particle according to the aspect of the present disclosure may include a blocking agent that covers at least part of the organic film and that has a property of inhibiting interaction between the organic film and a predetermined molecule.

Accordingly, when the analyte is detected, nonspecific adsorption between particles and nonspecific adsorption of impurities and the like to the surfaces of the particles can be reduced. Consequently, noise caused by nonspecific adsorption (that is, nonspecific adsorption noise) is reduced, and the analyte can be detected with high accuracy.

For example, in the modified particle according to the aspect of the present disclosure, the organic film may be a self-assembled monolayer.

Accordingly, the specific binding substance and the amino sugar molecule can readily bind to the self-assembled organic film. Consequently, the specific binding substance and the amino sugar molecule can be readily and stably fixed to the surface of the particle.

For example, in the modified particle according to the aspect of the present disclosure, the base material may contain a fluorophore.

Accordingly, a modified particle that has formed a specific bond with the analyte can be detected by an optical technique using fluorescence.

For example, in the modified particle according to the aspect of the present disclosure, the base material may contain a paramagnetic material or a dielectric material.

Accordingly, a modified particle that has formed a specific bond with the analyte can be detected by, for example, a technique to cause movement by using a magnetic field or an electric field.

Meanwhile, a method for manufacturing modified particles according to an aspect of the present disclosure includes preparing particles, fixing a specific binding substance that has a property of specifically binding to an analyte to the surfaces of the particles, and fixing an amino sugar molecule to the surfaces of the particles by using an amide bond.

Accordingly, regarding the resulting modified particles, even when the particles are in a storage liquid, deterioration of the specific binding substance can be reduced, and adsorption between the particles during detection or between the particle and a section with which the particle may come into contact during detection can be reduced.

For example, in the method for manufacturing modified particles according to the aspect of the present disclosure, the fixing of the specific binding substance and the fixing of the amino sugar molecule may be performed by mixing a solution containing the specific binding substance and the amino sugar molecule with the particle.

Accordingly, the fixing of the specific binding substance and the fixing of the amino sugar molecule can be performed in one step (simultaneously), and the number of steps during production can be decreased.

Meanwhile, a detection apparatus according to an aspect of the present disclosure includes a container for storing the modified particle described above, an introducer for introducing, to the container, a sample that may contain an analyte to which the modified particle specifically binds, and a detector for outputting a detection signal based on the amount of the analyte to which the modified particle has bound.

Accordingly, a detection apparatus that can detect the presence of the analyte or the amount of the analyte present in the sample by using the modified particle can be realized.

In this regard, the general or specific aspects may be realized as a system, a method, an integrated circuit, a computer program, or a recording medium such as CD-ROM which can be read by a computer or may be realized by any combination of a system, a method, an integrated circuit, a computer program, and a recording medium.

Embodiments of the present disclosure will be described below with reference to the drawings.

In this regard, each embodiment described below is a general or specific example. The numerical values, the shapes, the materials, the constituent elements, arrangement positions and connection forms of the constituent elements, the steps, the order of the steps, and the like described in the embodiments below are examples and are not intended to limit the claims. Of the constituent elements in the embodiments below, the constituent elements not described in an independent claim which illustrates the broadest concept are explained as optional constituent elements.

Each drawing is not necessarily precisely illustrated. In the drawings, substantially the same components are indicated by the same references, and duplicate explanations will be omitted or simplified.

In the present specification, words such as “parallel” indicating the relationship between elements, words such as “rectangular” indicating the shape of an element, numerical values, and ranges of numerical values are not expressions that represent strict meanings only but are expressions representing meanings within the range of substantially the same meaning, for example, including differences such as an error of several percent.

### Embodiment

### Configuration of Modified Particle

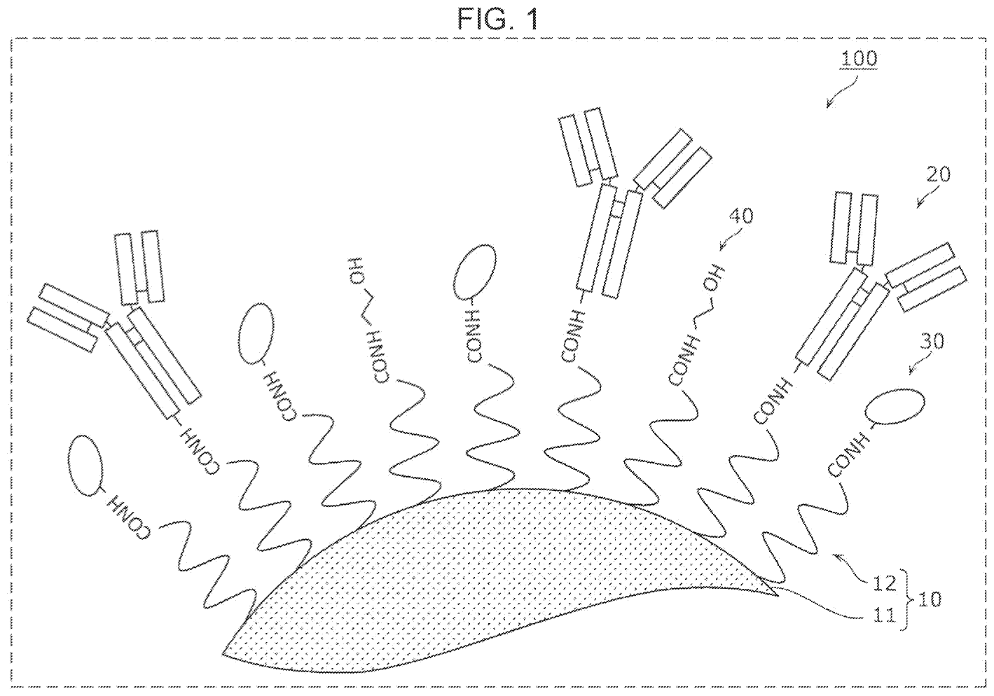

FIG. 1 is a schematic diagram illustrating an example of a modified particle 100 according to the present embodiment. FIG. 1 illustrates a cross section of the modified particle 100, where part of the modified particle 100 is illustrated.

As illustrated in FIG. 1, the modified particle 100 includes a particle 10, a specific binding substance 20, and an amino sugar molecule 30. The specific binding substance 20 has a property of specifically binding to the analyte and has been fixed to the surface of the particle 10. The amino sugar molecule 30 has been fixed to the surface of the particle 10 with an amide bond.

There is no particular limitation regarding the size of the particle 10 provided that the surface can be bound to the specific binding substance 20 and the amino sugar molecule 30.

Regarding the size of the particle 10, for example, the diameter is greater than or equal to 1 nm and less than or equal to 10 μm. A carboxy group is introduced onto the surface of the particle by using a known surface treatment technology. Consequently, the surface of the particle 10 can be bound to the amino sugar molecule 30. A particle to which a carboxy group is introduced also has the advantage of higher bindability to a specific binding substance 20 such as an antibody, compared with a particle to which an amino group is introduced.

In addition, the particle 10 includes a base material 11 and an organic film covering at least part of the base material 11. In particular, the surface of the particle 10 may be composed of molecules (linkers) that can appropriately ensure the distance between the specific binding substance 20 and the base material 11 from the viewpoint of ease of fixing the specific binding substance 20 and from the viewpoint of reactivity between the specific binding substance 20 and the analyte. The linker is composed of the organic film but is not limited to being composed of the organic film. The molecule that may serve as the linker is usually selected in accordance with the charge characteristics and the like of the surface to which the linker is bound.

The molecule that may serve as the linker in the present embodiment is composed of a molecule that forms a self-assembled monolayer (SAM 12), for example, an alkane thiol, but is not limited to this. In accordance with the characteristics of the base material 11, examples include silane coupling agents, hydrophilic polymers containing a polyethylene glycol chain (PEG chain), and MPC polymers which are polymers of 2-methacryloyloxyethyl phosphorylcholine (MPC) having a phospholipid polar group. Meanwhile, when these linker molecules are bound to the surface of the base material 11, binding may be performed through a metal or the like formed on the surface of the base material 11.

Regarding the metal material, at least one metal, for example, gold, silver, aluminum, copper, or platinum, or an alloy thereof may be used. In this regard, the metal material is not limited to these.

Examples of the material for forming the base material 11 include inorganic materials such as quartz, glass, silica, and ceramics, resins such as polystyrenes, polycarbonates, and cycloolefin polymers, natural materials, including rubber materials, such as hydrogel, agarose, cellulose, and isoprene, and metal materials such as iron, gold, aluminum, and silver.

The base material 11 may be formed so as to contain a fluorophore. The fluorophore is a substance which radiates fluorescence having a wavelength different from the wavelength of excitation light when irradiated with the excitation light, and, for example, organic coloring agents represented by fluorescein and derivatives thereof and biochemical fluorescent molecules such as green fluorescent protein may be used. Alternatively, quantum dots may be used as the fluorophore, wherein light emission characteristics of the radiofluorescence can be designed.

The base material 11 may be formed so as to contain a paramagnetic material or a dielectric material. For example, iron oxide and the like may be used as the paramagnetic material, and polystyrenes and the like may be used as the dielectric material, although these are not limitative.

As described above, in the present embodiment, the organic film is composed of the SAM 12 that may serve as the linker. At this time, the specific binding substance 20 has been fixed to the surface of the particle 10 by being bound to the SAM 12. In addition, the amino sugar molecule 30 has been fixed to the SAM 12 with an amide bond. Consequently, the particle 10 including the SAM 12 on the base material 11 stably fixes the specific binding substance 20 and the amino sugar molecule 30 to the surface of the particle 10. The amino sugar molecule 30 stably fixed to the surface of the particle 10 is not separated from the surface even when being subjected to a step of washing the modified particle 100 and the like. Since a hydroxy group (OH group) of the amino sugar molecule 30 acts instead of a water molecule, even when the solution containing the modified particle 100 is dried, deterioration of the specific binding substance due to drying is reduced, and higher detection sensitivity in detection of the analyte in which the modified particle 100 is actually used can be realized. In addition, since the stability of the specific binding substance 20 is increased as described above, ease of handling the modified particle 100 is improved.

In the present embodiment, as described above, the organic film is composed of linker molecules constituting the SAM 12. Regarding the monomolecule for forming the SAM 12, for example, carboxyalkanethiols having greater than or equal to about 4 and less than or equal to about 20 carbon atoms, in particular, 10-carboxy-1-decanethiol, may be used. An SAM 12 formed by using a carboxyalkanethiol having greater than or equal to about 4 and less than or equal to about 20 carbon atoms has properties such as high transparency, low refractive index, and small film thickness (that is, the distance from the surface of the base material 11 to the surface of the particle 10). Consequently, optical influence on detection by using the modified particle 100 is low. It is sufficient that one end of the SAM 12 is a functional group bindable to the surface of the base material 11, and in the case in which the one end is, for example, a thiol group, binding to gold present on the surface of the base material 11 forms the particle 10. Meanwhile, it is sufficient that the other end of the SAM 12 includes a carboxy group bindable to the specific binding substance 20 or to the amino sugar molecule 30. Since the SAM 12 has a carboxy group at the end, as described above, the specific binding substance 20 or the amino sugar molecule 30 can readily bind to the SAM 12. Further, the amino sugar molecule 30 is fixed with an amide bond. As a result, the specific binding substance 20 and the amino sugar molecule 30 are stably fixed to the surface of the particle 10.

The specific binding substance 20 is a substance that has a property of specifically binding to the analyte. The analyte is, for example, a protein, a lipid, a saccharide, or a nucleic acid and is a molecular species which is produced by a detection target such as a virus particle, a microorganism, a bacterium, or the like or which constitutes the detection target. Examples of the specific binding substance 20 include an antibody for an antigen, a substrate, an enzyme for a coenzyme, a receptor for a hormone, Protein A or Protein G for an antibody, avidin for biotin, calmodulin for calcium, and lectin for saccharides. In the case in which the analyte is a nucleic acid, a (complementary strand) nucleic acid having a sequence has a property of specifically binding to the nucleic acid may be used as the specific binding substance 20.

For example, in the case in which the specific binding substance 20 is a protein such as an antibody, some of a plurality of amino acids constituting the protein have a carboxy group, an amino group, or a thiol group in a side chain. These functional groups may be chemically bound to the particle 10. Alternatively, these functional groups may be modified with avidin, the particle 10 may be modified with biotin, and thereafter the specific binding substance 20 may be bound to the particle 10 by an avidin-biotin bond. In this regard, in the case in which the specific binding substance 20 is a protein, an amino group located at the N-terminal and a carboxy group located at the C-terminal may be used.

To more efficiently bind the specific binding substance 20 to the particle 10, activation treatment of the functional groups may be performed by using a substance for facilitating a binding reaction between the specific binding substance 20 and the particle 10. Regarding the activation treatment method, for example, 1-ethyl-3-(3-dimethylaminopropyl)carbodiimide hydrochloride (EDC) and N-hydroxysuccinimide (NHS) may be used. In the method, carboxy groups included in one of the SAM 12 and the specific binding substance 20 are subjected to active esterification, and the carboxy groups subjected to active esterification are bound to amino groups included in the other of the SAM 12 and the specific binding substance 20. Alternatively, the activation treatment method may be a method in which amino groups of the SAM 12 are bound to amino groups of the specific binding substance 20 by using a substance having a plurality of aldehyde groups, for example, glutaraldehyde.

In this regard, other known methods may be used as the method for fixing the specific binding substance 20 to the particle 10 provided that the fixing method does not deactivate the specific binding substance 20 (the specific binding ability is not lost).

The amino sugar molecule 30 is a saccharide having an amino group. The amino sugar molecule 30 has an amino group in the molecule and, thereby, is fixed to the surface of the particle 10 with an amide bond. In the present embodiment, the particle 10 has the SAM 12 (organic film) on the surface and has a carboxy group. Therefore, the amino group included in the amino sugar molecule 30 and the carboxy group included in the SAM 12 form an amide bond on the basis of a reaction between the carboxy group and the amino group. Consequently, the amino sugar molecule 30 is fixed to the SAM 12 with a covalent bond that is an amide bond. Among chemical bonds, the covalent bond has a strong binding force. As a result, the amino sugar molecule 30 is more stably fixed to the surface of the particle 10. Since the water retention of the thus stably fixed amino sugar molecule 30 protects the surface of the modified particle 100 from drying and reduces structural and functional deterioration of the specific binding substance 20, the storage stability of the modified particle 100 can be enhanced.

Examples of the amino sugar molecule 30 include not only monosaccharides but also disaccharides, oligosaccharides composed of three or more monosaccharides, and polysaccharides (so-called glycans). The amino sugar molecule 30 may have a functional group other than the amino group in one molecule in the same manner as sialic acid. Alternatively, the amino sugar molecule 30 may be a salt of an amino sugar molecule as given above. Preferable examples of the amino sugar molecule 30 include monosaccharides and disaccharides.

Specifically, the amino sugar molecule 30 may be, for example, an amino sugar having an amino group such as glucosamine, mannosamine, galactosamine, sialic acid, aminouronic acid, or muramic acid, a polysaccharide having an amino group such as chitosan, or a salt thereof. In this regard, in the case in which there is a D- or L-enantiomer of the above-described amino sugar molecule 30, either may be used. Preferably, the amino sugar molecule 30 is glucosamine.

Regarding the method for fixing the amino sugar molecule 30 to the surface of the particle 10, the same method as for the specific binding substance 20 may be used. There is no particular limitation regarding the amino sugar molecule 30 provided that the amino sugar molecule can be fixed to the surface of the particle 10 with an amide bond, and known saccharides other than the above-described saccharides may be used.

It can be determined that the amino sugar molecule 30 has been fixed to the surface of the particle 10 with an amide bond, based on the presence of an infrared absorption peak derived from the amino sugar molecule 30 in a decomposition product obtained by subjecting the modified particle 100 to the action of a protease such as trypsin.

The modified particle 100 according to the present embodiment may further contain a blocking agent that covers at least part of the surface of the particle 10 and that has a property of inhibiting interaction of the SAM 12 of the modified particle 100 with other molecules. The blocking agent is a substance for inhibiting interaction (that is, nonspecific adsorption) in which impurities and the like in a sample that may contain an analyte nonspecifically adsorb or bind to the surface of the particle 10. Examples of the impurities include predetermined molecules such as proteins, lipids, saccharides, peptides, and nucleic acids other than molecules constituting the modified particle.

In the present embodiment, the blocking agent is described as ethanolamine 40 but is not limited to this. Examples of the blocking agent include skim milk, fish gelatin, bovine serum albumin (BSA), surfactants, casein, protamine, and polyethylene glycol (PEG). It is sufficient that the blocking agent covers at least a region of the surface of the particle 10 in which neither the specific binding substance 20 nor the amino sugar molecule 30 is fixed (that is, a gap region).

When the analyte is detected, such a blocking agent can inhibit nonspecific adsorption to the surface of the particle 10. Consequently, noise caused by nonspecific adsorption (that is, nonspecific adsorption noise) is reduced, and higher detection sensitivity can be realized when the analyte is detected.

In the present disclosure, blocking treatment is treatment to inhibit nonspecific adsorption as described above. The blocking treatment can reduce the influence of nonspecific adsorption on detection of the analyte. In the blocking treatment, it is preferable that the specific binding substance 20 and the amino sugar molecule 30 be fixed to the surface of the particle 10 and thereafter that the ethanolamine 40 be fixed to the surface of the particle 10.

More specifically, after the specific binding substance 20 and the amino sugar molecule 30 are fixed to the particle 10, a solution containing the ethanolamine 40 is added so as to fix the ethanolamine 40 in the solution to the surface of the particle 10. The solution containing the ethanolamine 40 is subjected to a reaction for a predetermined time (for example, a reaction time for sufficiently covering the gap region) and is removed by external solution exchange or the like since excess (unfixed) ethanolamine 40 is contained. In this regard, in the case in which the blocking agent is sufficiently fixed by the reaction, the blocking treatment may be performed simultaneously with fixing of the specific binding substance 20 and the amino sugar molecule 30.

### Method for Manufacturing Modified Particle

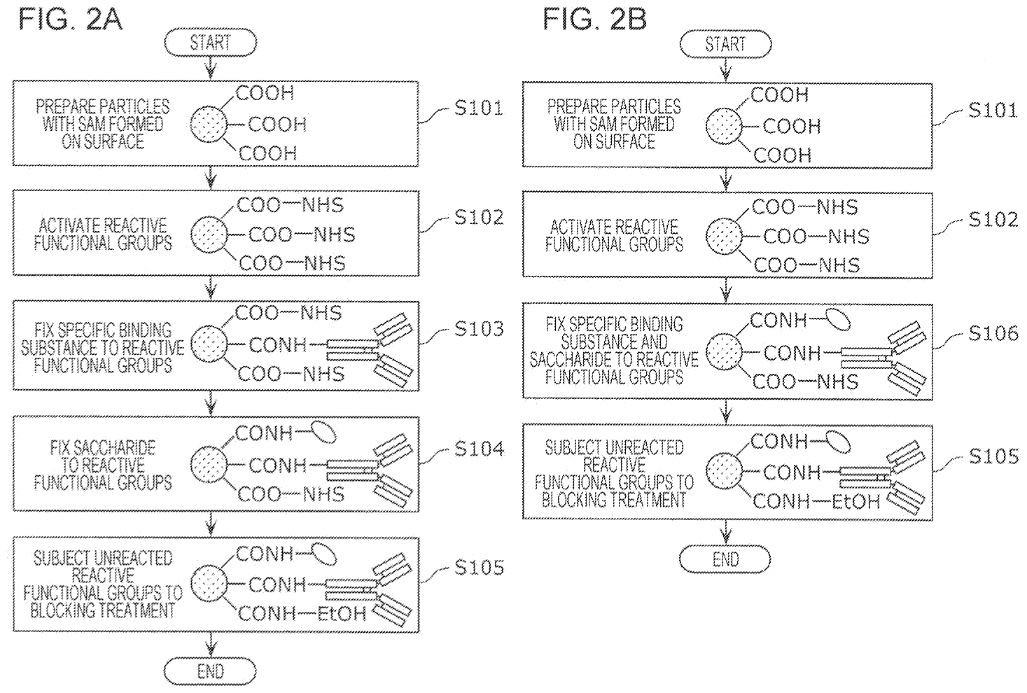

FIGS. 2A and 2B are flow charts illustrating an example of a method for manufacturing modified particles 100 according to the present embodiment.

As illustrated in FIG. 2A, in the method for manufacturing the modified particles 100, (1) a preparation step (S101) of preparing particles 10 and (2) an activation step (S102) of activating reactive functional groups are performed. In this regard, the activation step is performed to increase the reaction efficiency of a fixing step and a saccharide fixing step following the activation step. Therefore, for example, in the case in which reactive functional groups having sufficient reactivity are introduced in accordance with selection of the reactive functional groups or the like, the activation step may be unnecessary.

In addition, the method for manufacturing the modified particles 100 includes (3) a fixing step (S103) of fixing the specific binding substance 20 that has a property of specifically binding to the analyte to the surface of the particle 10 and (4) a saccharide fixing step (S104) of fixing the amino sugar molecule 30 to the surfaces of the particles 10 by using an amide bond.

Further, (5) a blocking step (S105) of performing blocking treatment may be performed in the method for manufacturing the modified particles 100 since it is possible that unreacted reactive functional groups contribute to nonspecific adsorption. In this regard, when the conditions of the fixing step and the saccharide fixing step under which unreacted reactive functional groups are not formed (do not remain) can be set, the blocking step does not have to be performed.

The method for manufacturing the modified particles 100 will be more specifically described below.

(1) The preparation step (S101) in the method for manufacturing the modified particles 100 includes, for example, three sub-steps below. A first sub-step is a step of preparing the base material 11. A second sub-step is a step of forming the SAM 12 on the base material 11.

Each sub-step will be more specifically described below.

In the first sub-step, for example, in the case in which the material for forming the base material 11 is a resin material, the base material 11 is formed by using a known synthesis technology such as polymerization. Meanwhile, in the case in which the material for forming the base material 11 is a metal or a paramagnetic material, the base material 11 is also formed by using a known synthesis technology.

Subsequently, in the second sub-step, the SAM 12 is formed on the surface (for example, the surface on which a metal is formed) of the base material 11. There is no particular limitation regarding the method for forming the SAM 12, and a commonly used method may be used. Examples of the method include a method in which the base material 11 with the metal formed on the surface is immersed in an ethanol solution containing a carboxyalkanethiol having greater than or equal to about 4 and less than or equal to about 20 carbon atoms (for example, 10-carboxy-1-decanethiol).

In this method, thiol groups of the carboxyalkanethiol (hereafter referred to as monomolecules) binding to the metal fixes the monomolecules to the surface of the metal base material, and the fixed monomolecules undergo self-assembly due to interaction on the surface of the metal so as to form a film. In this manner, particles 10 in which the SAM 12 is arranged on the surface of the base material 11 are obtained.

(2) The activation step (S102) is a step of activating reactive functional groups (for example, carboxy groups) introduced on the particle-10-surface side of the SAM 12 into a form that readily reacts with the reactive functional groups (for example, amino groups) included in the specific binding substance 20. In the present activation step, for example, in the case in which the monomolecules constituting the SAM 12 have a carboxy group, active esterification is performed with 1-ethyl-3-(3-dimethylaminopropyl)carbodiimide (EDC) and N-hydroxysuccinimide (NHS). In the present activation step, the reactive functional groups are changed to a more reactive state by the above-described modification or by a reaction such as elimination that is not described in the example.

(3) The fixing step (S103) includes, for example, three sub-steps below. A fourth sub-step is a step of mixing the specific binding substance 20 with the particles 10. A fifth sub-step is a step of fixing the specific binding substance 20 to the SAM 12 (that is, to the particles 10). A sixth sub-step is a step of removing free specific binding substance 20 that has not been fixed to the SAM 12.

Each sub-step will be more specifically described below.

In the fourth sub-step, the particles 10 including the SAM 12 in which the reactive functional groups are activated in the step S102 are mixed with a solution containing the specific binding substance 20.

In the fifth sub-step, the reaction between the activated reactive functional groups included in the SAM 12 and the reactive functional groups included in the specific binding substance 20 is performed so as to fix the specific binding substance 20 to the SAM 12. The specific binding substance 20 is fixed to the SAM 12 with an amide bond.

In the sixth sub-step, free specific binding substance 20 that has not been fixed to the SAM 12 is removed. More specifically, free molecules are removed by a method such as external solution exchange while an environment in which fixed reactive functional groups are not decomposed is maintained by using a buffer solution, for example, a phosphate buffered saline (PBS) solution. Consequently, modified particles 100 in which the specific binding substance 20 has been fixed to the surfaces of the particles 10 can be obtained.

(4) The saccharide fixing step (S104) includes, for example, sub-steps as in the above-described fixing step. That is, sub-steps corresponding to the fourth sub-step to the sixth sub-step are included such that the amino sugar molecule 30 instead of the specific binding substance 20 is fixed.

In this regard, as illustrated in FIG. 2B, the fixing step and the saccharide fixing step may be simultaneously performed. More specifically, the reactive functional groups are activated in the activation step, and thereafter the activated particles 10 and a solution containing the specific binding substance 20 and the amino sugar molecule 30 are mixed. Consequently, the fifth sub-step and the sub-step in the saccharide fixing step corresponding to the fifth sub-step are simultaneously performed in one step (S106).

That is, in the present manufacturing method, the solution containing the specific binding substance 20 and the amino sugar molecule 30 and the particles 10 having activated reactive functional groups may be mixed, and the fixing step (S103) of fixing the specific binding substance 20 to the SAM 12 formed on the surfaces of the particles 10 and the saccharide fixing step (S104) of fixing the amino sugar molecule 30 may be simultaneously performed.

The method for manufacturing the modified particles 100 may further include (5) the blocking step (S105). In the blocking step, as described above, treatment to cover the surfaces of the particles 10 with the ethanolamine 40 (blocking agent) for inhibiting impurities and the like in the sample that may contain the analyte from nonspecifically adsorbing or binding to the surfaces of the particles 10 is performed.

Specifically, in the blocking step, the ethanolamine 40 and the particles 10 to which the specific binding substance 20 and the amino sugar molecule 30 are fixed are mixed. Consequently, the modified particles 100 containing the ethanolamine 40 are obtained, and nonspecific adsorption of impurities and the like in the sample to the surfaces of the particles 10 can be reduced.

Further, a solution may be prepared by adding a blocking agent to a solution containing the specific binding substance 20 and the amino sugar molecule 30, and the particles 10 with activated reactive functional groups may be placed into and mixed with the resulting solution. Consequently, the fixing step, the saccharide fixing step, and the blocking step may also be simultaneously performed in one step. Therefore, the manufacturing steps can be further combined so as to decrease the number of steps in the production of the modified particles 100.

### Configuration of Detection Apparatus

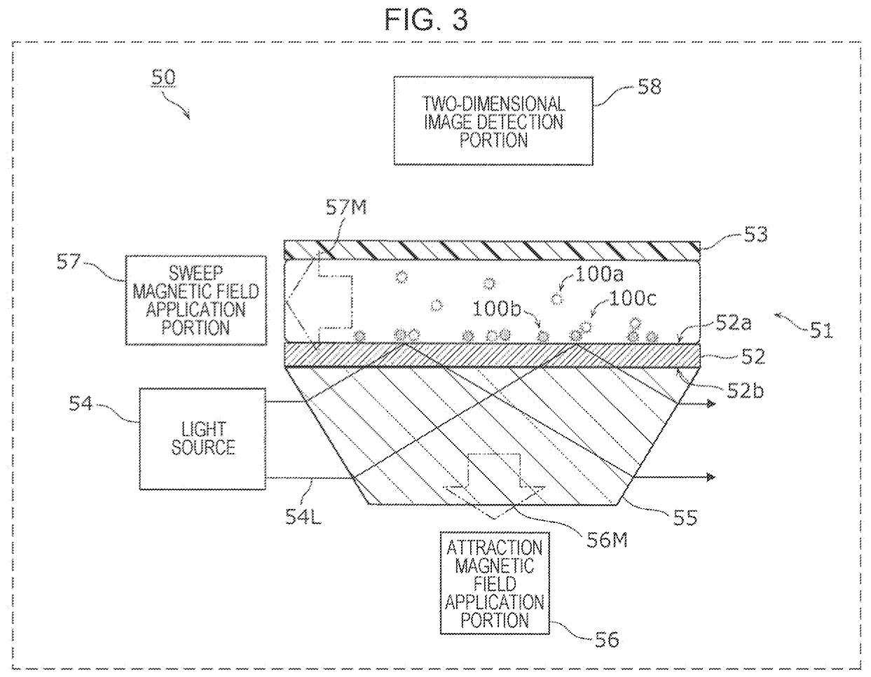

FIG. 3 is a schematic configuration diagram illustrating an example of a detection apparatus 50 according to the present embodiment.

As illustrated in FIG. 3, the detection apparatus 50 includes a container for storing the modified particle 100, an introducer for introducing, to the container, a sample that may contain an analyte to which the modified particle 100 specifically binds, and a detector for outputting a detection signal based on the amount of the analyte to which the modified particle 100 has bound.

In particular, the detection apparatus 50 includes a cell 51 (an example of the container) for storing the modified particle 100, a light source 54, an attraction magnetic field application portion 56, a sweep magnetic field application portion 57, and a two-dimensional image detection portion 58 (an example of the detector). The cell 51 has a space delimited by a detection plate 52 and a cover 53, and a prism 55 is bonded to the surface of the detection plate 52 opposite to the surface facing the space. In this regard, the space of the cell 51 is formed so that access to the outside is made possible by, for example, opening the cover 53. That is, in this example, the cover 53 functions as the introducer.

Meanwhile, the configuration may include a communication hole (not illustrated in the drawing) that can introduce the sample which may contain the analyte into the cell 51. In such an example, the communication hole functions as the introducer. Therefore, the introducer may be included in any of the constituent elements of the detection apparatus 50 provided that the sample which may contain the analyte can be introduced.

Each constituent element of the detection apparatus 50 will be described below in detail. In an apparatus described as an example of the detection apparatus 50 in the present embodiment, the analyte is detected by using a detection method called external force-assisted near-field illumination (EFA-NI).

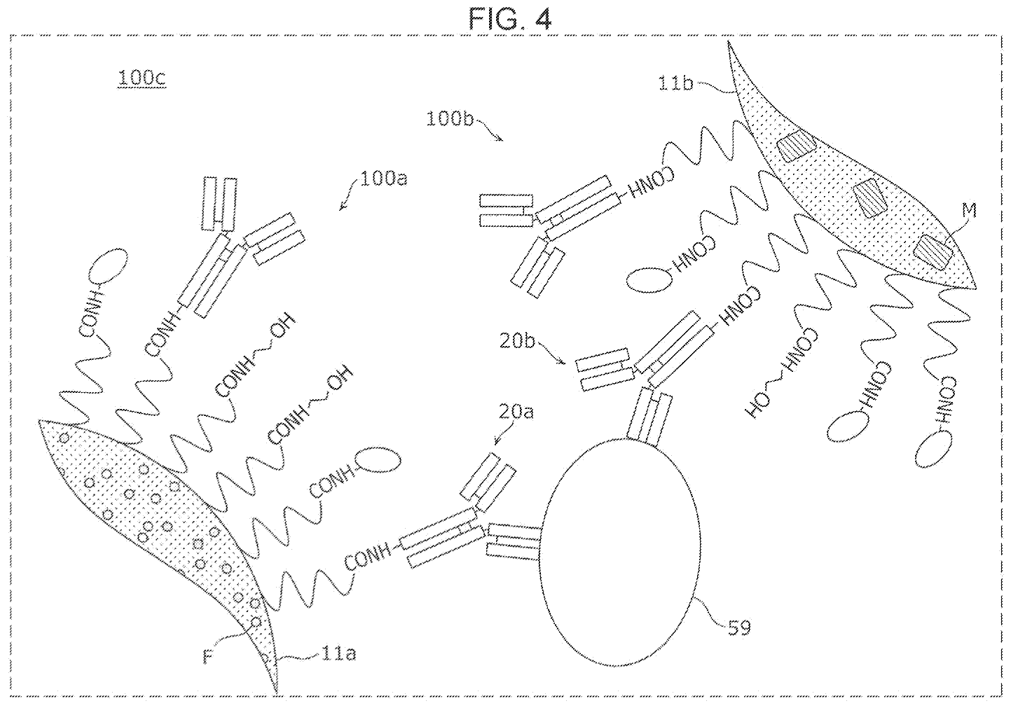

Regarding detection of the analyte by using the detection apparatus 50, at least two types of modified particles 100 are used. Such particles will be described with reference to FIG. 4. FIG. 4 is a diagram illustrating modified particles when used for the detection apparatus 50. The modified particles 100 in FIG. 4 are illustrated in a sectional view, where part of each modified particle 100 is omitted from the drawing as in FIG. 1.

As illustrated in FIG. 4, when an analyte 59 is detected by the detection apparatus 50, a modified particle in which a base material 11a contains a fluorophore F (that is, a first particle 100a) and a modified particle in which a base material 11b contains a paramagnetic material M (that is, a second particle 100b) are used. The first particle 100a binds to the analyte 59 with a specific binding substance 20a interposed therebetween. Likewise, the second particle 100b binds to the analyte 59 with a specific binding substance 20b interposed therebetween. In this regard, the specific binding substance 20a and the specific binding substance 20b bind to different positions of the analyte 59.

For example, in the case in which the analyte 59 is a molecule having a repeating structure such as a virus coat protein, the specific binding substance 20a and the specific binding substance 20b may bind to identical positions in the molecule. In particular, even when the specific binding substance 20a binds to one binding position in the repeating structure, since there are a plurality of identical binding positions in the repeating structure, the specific binding substance 20b can simultaneously bind to an identical binding position.

On the other hand, in the case in which the analyte 59 is a single molecule having no identical binding positions, such as a single protein molecule, the specific binding substance 20a and the specific binding substance 20b need to bind to different positions in the molecule of the analyte 59. Therefore, the modified particle needs to be designed so that, in particular, the binding positions of the specific binding substance 20a and the specific binding substance 20b with respect to the analyte 59 have the same structure or different structures in accordance with the type of analyte 59.

The two types of modified particles (the first particle 100a and the second particle 100b) designed in consideration of the above bind to the analyte 59 so as to form a particle complex 100c.

Referring to FIG. 3 again, there is illustrated a state in which the first particles 100a, the second particles 100b, and the analyte 59 are introduced into the space in the cell 51 and in which some of these form the particle complexes 100c.

As described above, the cell 51 is delimited by the detection plate 52 that is a tabular member. Therefore, one principal surface 52a of the detection plate 52 faces the space of the cell 51. The other principal surface 52b of the detection plate 52 faces the prism 55, and they are bonded to each other. Herein, the detection plate 52 is irradiated with excitation light 54L from the other-principal-surface-52b side. The excitation light 54L passes through the light-transmitting prism 55 and is further incident on the other principal surface 52b of the detection plate 52. The excitation light 54L incident on the other principal surface 52b of the detection plate 52 passes through the detection plate 52 and is reflected by the one principal surface 52a of the detection plate 52. Thus, the arrangements, the refractive indices, the interfacial shapes, and the like of the detection plate 52, the prism 55, and the light source 54 for radiating the excitation light 54L are designed such that the excitation light 54L is totally reflected by the one principal surface 52a of the detection plate 52.

As described above, the excitation light 54L is totally reflected by the one principal surface 52a of the detection plate 52 and, at this time, forms a near field such as an evanescent field or an enhanced electric field in the vicinity of the one principal surface 52a of the detection plate 52 in the space of the cell 51. The near field is formed in the vicinity of the one principal surface 52a only and has a property of rapidly attenuating with increasing distance from the one principal surface 52a of the detection plate 52. Therefore, only the space of the cell 51 in the vicinity of the one principal surface 52a of the detection plate 52 is irradiated. There is no particular limitation regarding the configuration of the detection plate 52, and the configuration may be appropriately selected in accordance with purpose. The detection plate 52 may be composed of a single layer or may be composed of a multilayer body for the purpose of enhancing the electric field.

As described above, the light source 54 is an example of a light-radiation portion that emits light of a predetermined wavelength to form a near field so that the space of the cell 51 is irradiated. Regarding the light source 54, known technologies may be used with no particular limitation. For example, a laser such as a semiconductor laser or a gas laser may be used as the light source 54. In this regard, it is preferable that the light source 54 radiate excitation light of a wavelength (for example, 400 nm to 2,000 nm) having a small interaction with a substance contained in the analyte 59. Further, the wavelength of the excitation light is preferably 400 nm to 850 nm for which a semiconductor laser can be used.

The cover 53 is a light-transmitting tabular member facing the one principal surface 52a of the detection plate 52 and is formed by using any material such as a resin. The cover 53 is disposed at a predetermined distance from the detection plate 52, and the volume of the space of the cell 51 can be changed in accordance with the distance. Therefore, the distance between the detection plate 52 and the cover 53 is appropriately set in accordance with the use to which the detection apparatus 50 is applied.

A two-dimensional image detection portion 58 is arranged at a distance from the surface of the cover 53 opposite to the one principal surface facing the space of the cell 51 and detects light that is generated in the space of the cell 51 by irradiation with the excitation light 54L and that is made to form a two-dimensional image.

The attraction magnetic field application portion 56 generates a first magnetic field gradient 56M indicated by a double-dotted chain line arrow in the drawing in the space of the cell 51. The attraction magnetic field application portion 56 is composed of an electromagnet capable of switching between ON and OFF but may have a configuration in which a permanent magnet is moved toward or away from the space of the cell 51. The first magnetic field gradient 56M is applied to the space of the cell 51 by the attraction magnetic field application portion 56, and a paramagnetic material present in the space of the cell 51 is attracted toward the prism 55 in a direction perpendicular to the detection plate 52.

The sweep magnetic field application portion 57 generates a second magnetic field gradient 57M indicated by a double-dotted chain line arrow in the drawing in the space of the cell 51. As with the attraction magnetic field application portion 56, the sweep magnetic field application portion 57 is composed of an electromagnet but may have a configuration in which a permanent magnet is moved toward or away from the space of the cell 51. The second magnetic field gradient 57M is applied to the space of the cell 51 by the sweep magnetic field application portion 57, and a paramagnetic material present in the space of the cell 51 is attracted toward the light source 54 in a direction parallel to the detection plate 52. In this regard, the sweep magnetic field application portion 57 may be arranged at any position in a direction parallel to the detection plate 52. In the following explanation, the above-described arrangement is used as an example.

That is, the attraction magnetic field application portion 56 and the sweep magnetic field application portion 57 are examples of the magnetic field application portion for applying a magnetic field to the space of the cell 51.

### Method for Detecting Analyte

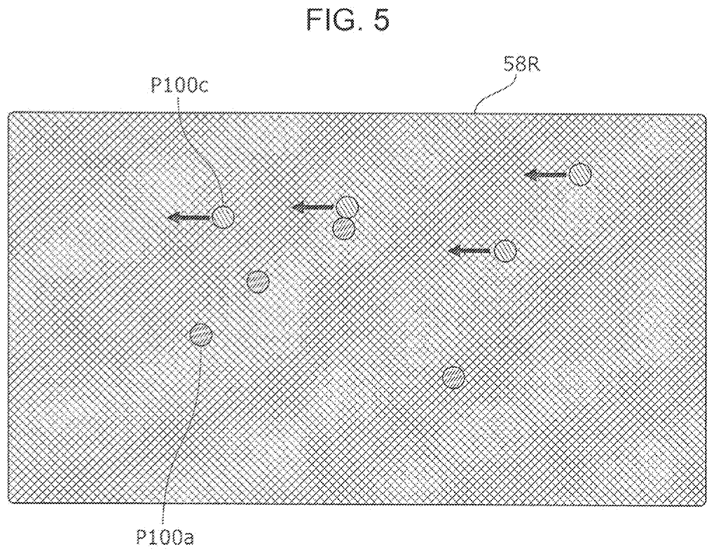

Next, the method for detecting the analyte 59 by using the detection apparatus 50 having the above-described configuration according to the present embodiment will be described with reference to FIG. 5. FIG. 5 is a schematic diagram illustrating a two-dimensional image output from the detection apparatus 50 according to the present embodiment.

The space of the cell 51 stores the first particle 100a and the second particle 100b in advance.

A sample that may contain the analyte 59 is introduced therein. The analyte 59 contained in the sample is bound to the first particle 100a and the second particle 100b so as to form the particle complex 100c in the space of the cell 51.

In this regard, the first particle 100a and the second particle 100b are particles in which the amino sugar molecule 30 has been fixed to the surface. Such a configuration suppresses adsorption of the first particle 100a and the second particle 100b to the detection plate 52 and suppresses adsorption of the first particle 100a and the second particle 100b to each other. For example, if the first particle 100a adsorbs to the detection plate 52, since irradiation with a near field occurs and background light is enhanced, the detection sensitivity deteriorates, as described later. For example, if the first particle 100a and the second particle 100b adsorb to each other, since their behavior is similar to that of the particle complex 100c despite having bound to no analyte 59, errors are caused in counting the analyte 59.

However, the amino sugar molecule 30 being fixed to the first particle 100a and the second particle 100b enables deterioration of the detection sensitivity to be reduced and enables causes of errors to be reduced.

Since the particle complex 100c contains the second particle 100b, the paramagnetic material M contained in the base material 11b is attracted by the applied magnetic field. Consequently, when the first magnetic field gradient 56M is applied to the space of the cell 51 by the attraction magnetic field application portion 56, the particle complex 100c is attracted so as to take on a state of being substantially in contact with the detection plate 52. Likewise, the second particle 100b that does not form the particle complex 100c present in the space of the cell 51 are also attracted so as to take on a state of being substantially in contact with the detection plate 52. Meanwhile, the first particle 100a that does not form the particle complex 100c is not attracted from the original state because the paramagnetic material M is not contained.

When the excitation light 54L is emitted from the light source 54, as described above, a near field is formed in the vicinity of the one principal surface 52a of the detection plate 52. The particle complex 100c includes the bound first particle 100a and, therefore, emits fluorescence when irradiated with light having a wavelength that excites a fluorophore F contained in the base material 11a. That is, when the near field is light having the excitation wavelength of the fluorophore F, the particle complex 100c emits fluorescence. Meanwhile, the first particle 100a that does not form the particle complex 100c also emits fluorescence when irradiated with the near field.

However, the near field is formed in the vicinity of the one principal surface 52a of the detection plate 52 only. That is, of the first particles 100a, the first particle 100a in the state of being substantially in contact with the detection plate 52 can emit fluorescence. Since the first particle 100a does not contain the paramagnetic material M, only a small proportion of the total first particles 100a are irradiated with the near field as described above.

In this manner, the particle complexes 100c and some of the first particles 100a emit fluorescence in the space of the cell 51.

Herein, FIG. 5 illustrates a two-dimensional image 58R output from the two-dimensional image detection portion 58 in the above-described situation. In the output two-dimensional image 58R, light spots P100c derived from fluorescence emitted from the particle complexes 100c and light spots P100a derived from fluorescence emitted from the first particles 100a are illustrated.

In this regard, the light spots P100c cannot be distinguished from the light spots P100a. Then, the second magnetic field gradient 57M is applied to the space of the cell 51 by the sweep magnetic field application portion 57. At this time, the above-described two-dimensional image 58R being continuously output enables changes caused by the second magnetic field gradient 57M to be obtained as a two-dimensional moving image.

As described above, the particle complex 100c contains the paramagnetic material M and, therefore, is attracted by the second magnetic field gradient 57M, but the first particle 100a does not have the paramagnetic material M and, therefore, remains at the original position. Consequently, movements of the light spots P100c indicated by arrows in FIG. 5 are observed in the resulting two-dimensional moving image. On the other hand, such a movement is not observed regarding the light spot P100a, and the particle complex 100c and the first particle 100a can be distinguished and counted due to this difference.

Therefore, when the analyte 59 to which the first particle 100a and the second particle 100b have bound (that is, the particle complex 100c) is moved by the first magnetic field gradient 56M and the second magnetic field gradient 57M, on the basis of the fluorescence emitted from the fluorophore F due to the near field having a predetermined wavelength, the two-dimensional image detection portion 58 outputs the two-dimensional image 58R which enables the analyte 59 to be counted. That is, the two-dimensional image 58R is an example of the detection signal based on the amount of the analyte 59 here.

In the configuration, such light spots may be automatically counted by image recognition of the two-dimensional image 58R output from the two-dimensional image detection portion 58.

### EXAMPLE

The modified particle according to the present disclosure will be specifically described below with reference to an example. However, the present disclosure is not limited to the following example only.

### EXAMPLE

In the example, an antibody for an antigen that was a nucleoprotein (NP) of influenza A virus was used as the specific binding substance. Glucosamine was used as the amino sugar. In the present example, the method illustrated in FIG. 2B was used as the method for manufacturing the modified particles. Therefore, the method in which the specific binding substance and the saccharide were bound in one step was adopted.

(i) Particles provided with SAM were subjected to external solution exchange by centrifugal separation so that the final particle concentration was 1 mg/mL in a 25 mM 2-morpholinoethanesulfonic acid (MES) buffered saline solution. Thereafter, an external solution fraction was discarded by performing centrifugal separation again.

(ii) After 60 μL of MES buffered saline solution containing 50 mg/mL of N-hydroxysuccinimide (NHS) and 50 mg/mL of 1-(3-dimethylaminopropyl)-3-ethylcarbodiimide hydrochloride (EDC) was added and left to stand, an external solution fraction was discarded by performing centrifugal separation.

(iii) Subsequently, 100 μL of 25 mM acetate buffer solution containing 0.5 mg/mL of the antibody and 1% of glucosamine was added, and a reaction was performed. Consequently, the antibody and glucosamine were fixed to the SAM. Thereafter, an external solution fraction was discarded by performing centrifugal separation.

(iv) Subsequently, 240 μL of 1 M ethanolamine solution containing 0.1% of Nonidet P-40 (NP-40) was added, and a reaction was performed.

(v) Further, an external solution fraction was discarded by performing centrifugal separation, and 200 μL of 10 mM 4-(2-hydroxyethyl)-1-piperazineethanesulfonic acid (HEPES) solution containing 50 mM of potassium chloride, 1 mM of ethylenediaminetetraacetic acid (EDTA), and 10% of glycerin was added. After the operation to discard an external solution fraction by performing centrifugal separation, 100 μL of the above-described solution was added.

The modified particles according to the example were obtained by the above-described steps. The modified particles according to the example included a particle, antibodies, glucosamine, and ethanolamine.

Meanwhile, methods for manufacturing modified particles according to three comparative examples will be described below.

### Comparative Example 1

The same steps of the method for manufacturing the modified particles according to the above-described example were performed except that the step of (iii) and the step of (iv) were not performed. Consequently, modified particles according to comparative example 1 were obtained. The modified particles according to comparative example 1 included a particle and antibodies and were different from the modified particles according to the example in that neither glucosamine nor ethanolamine was included.

### Comparative Example 2

The same steps of the method for manufacturing the modified particles according to the above-described example were performed except that 1% of glucosamine was not added in the step of (iii). Consequently, modified particles according to comparative example 2 were obtained. The modified particles according to comparative example 2 included a particle, antibodies, and ethanolamine and were different from the modified particles according to the example in that glucosamine was not included.

### Comparative Example 3

The same steps of the method for manufacturing the modified particles according to the above-described example were performed except that 1% of trehalose was added instead of 1% of glucosamine in the step of (iii). Consequently, modified particles according to comparative example 3 were obtained. The modified particles according to comparative example 3 included a particle, antibodies, and ethanolamine, and trehalose molecules that did not form a bond were present around the modified particles. Therefore, the modified particles according to comparative example 3 were different from the modified particles according to the example in that glucosamine was not included and in that trehalose molecules were present around the modified particles.

### Evaluation of Nonspecific Adsorption

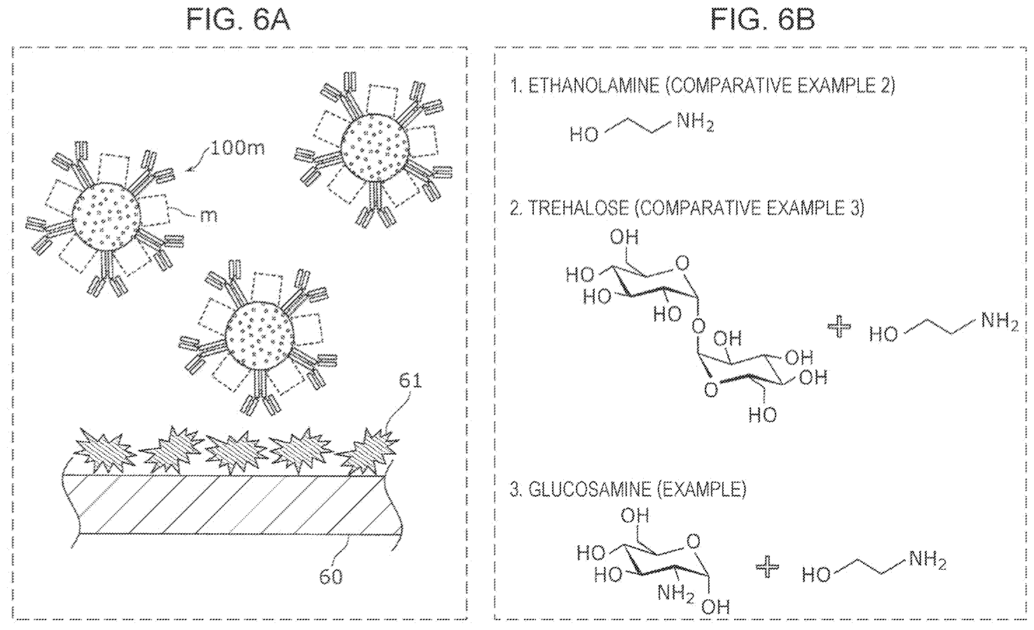

Next, the evaluation of nonspecific adsorption by using the modified particles according to the example will be described with reference to FIGS. 6A and 6B. FIGS. 6A and 6B are diagrams illustrating a test method for an adsorption test of the modified particles according to the example.

FIG. 6A is a schematic diagram illustrating a test method for evaluating nonspecific adsorption. In the present test method, human serum albumin (HSA 61) is fixed to the bottom surface 60 of a 96-well plate in advance, the amount of the modified particles adsorbed to the fixed HSA 61 is detected, and the degree of nonspecific adsorption is compared with that of the modified particles according to the comparative example.

The test method performed was as described below in detail. Initially, 50 μL of phosphate buffered saline solution containing 3.2 μM of HSA 61 and 0.5% of Tween (registered trademark) 20 was added to each well of the bottom surface 60 of the 96-well plate. A reaction was performed all night at 4° C. so as to fix the HSA 61. Thereafter, the phosphate buffered saline solution containing the HSA 61 and 0.5% of Tween (registered trademark) 20 was discarded. Modified particles in which the base material contained fluorophores were used, and 50 μL of modified particle solution in which the final particle concentration was 7×10⁹/mL was added to each well. Subsequently, a reaction was performed at room temperature for 60 minutes, and the modified particle solution was discarded.

Further, the treatment to add 200 μL of phosphate buffered saline solution containing 0.05% of Tween (registered trademark) 20 to each well and then discard the solution was performed three times to wash each well. Remaining modified particles that had bound to the HSA 61 were measured on the basis of fluorescence (under the conditions of excitation wavelength: 532 nm and fluorescence wavelength: 568 nm), and the fluorescence intensities of the example and comparative examples 1 to 3 were compared. In this regard, 4 wells of each of the example and comparative examples 1 to 3 were subjected to the test (n=4).

Since the fluorescence intensity decreases with decreasing adsorption (nonspecific adsorption) to the HSA 61, in the present adsorption test, lower fluorescence intensity indicates higher detection sensitivity to the analyte.

In this regard, the modified particle indicated by reference 100m in FIG. 6A represents any one of the modified particle according to the example and the modified particles according to comparative examples 1 to 3.

As illustrated in FIG. 6B, a molecule that differs in accordance with the modified particle 100m being the modified particle according to the example or the modified particle according to any one of comparative examples 1 to 3 is included at the modification position m indicated by a broken-line rectangle in the modified particle 100m. Regarding the modified particle according to comparative example 1, no molecule is introduced into the modification position m and, therefore, antibodies only are included as described above.

Regarding the modified particle according to comparative example 2, ethanolamine is included at the modification position m as illustrated in item 1 of FIG. 6B. The ethanolamine has been fixed to the SAM, although not illustrated in FIG. 6A.

Regarding the modified particle according to comparative example 3, trehalose and ethanolamine are included at the modification position m as illustrated in item 2 of FIG. 6B. The ethanolamine has been fixed to the SAM as in comparative example 1, but trehalose has not been fixed and is present around the modified particle.

Regarding the modified particle according to the example, glucosamine and ethanolamine are included at the modification position m as illustrated in item 3 of FIG. 6B. Each of the ethanolamine and glucosamine has been fixed to the SAM.

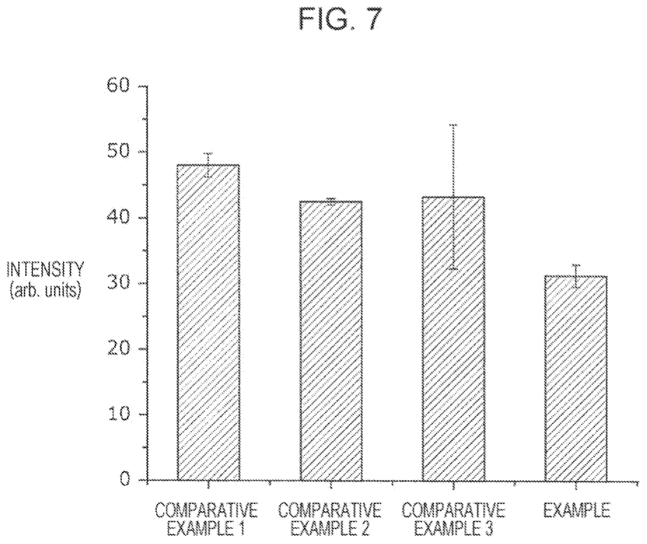

Next, the test results of the present adsorption test will be described with reference to FIG. 7. FIG. 7 is a diagram illustrating the results of the adsorption test of the modified particles according to the example.

In FIG. 7, the vertical axis represents the relative fluorescence intensity, and the horizontal axis represents each of the modified particles (comparative example 1, comparative example 2, comparative example 3, and example) arranged side by side. Each bar graph represents an average value at n=4, and an error of each measurement is also indicated by an error bar.

As illustrated in FIG. 7, the modified particle having antibodies only according to comparative example 1 exhibited the highest fluorescence intensity, and the modified particle according to comparative example 2 exhibited lower fluorescence intensity than the modified particle according to comparative example 1. That is, an effect of suppressing nonspecific adsorption due to introduction of ethanolamine was ascertained. In addition, as a result of comparison between comparative example 2 and the example, the fluorescence intensity of the modified particle according to the example was considerably low, and an effect of suppressing nonspecific adsorption due to fixing of glucosamine was ascertained.

Meanwhile, the modified particle according to comparative example 3 exhibited fluorescence intensity similar to that of comparative example 2, and it was found that only the effect of suppressing nonspecific adsorption due to introduction of ethanolamine was obtained. A possible reason for this is that trehalose was in the state of being not bound and was removed by washing. In addition, a possible reason for a large error in comparative example 3 is that some trehalose molecules were not removed by washing and remained because of high viscosity of trehalose.

According to the above results, the amino sugar molecule being bound with an amide bond improved the effect of suppressing nonspecific adsorption. Therefore, it was demonstrated that the sensitivity of detection of the analyte can be enhanced by using the amide bond of the amino sugar molecule.

The modified particles, the method for manufacturing the modified particles, and the detection apparatus according to the present disclosure have been described above with reference to the embodiment and the example, but the present disclosure is not limited to the embodiment and the example. Various modifications made to the embodiment and the example as conceived by a person skilled in the art and other embodiments constructed by combining some constituent elements of the embodiment and the example are included in the scope of the present disclosure without departing from the spirit of the present disclosure.

In this regard, the modified particles, the method for manufacturing the modified particles, and the detection apparatus according to the present disclosure may be used for, for example, a detection system that detects viruses floating in air.

Further, other embodiments will be described below,

### Other Embodiments

In the embodiment above, the configuration in which the modified particle 100 includes the SAM 12 as an organic film has been described. However, the SAM 12 need not be included if the specific binding substance 20 and the amino sugar molecule 30 can bind to the base material 11. In this regard, for example, a method in which a resin having a reactive functional group is used as a base material 11 or a method in which a base material 11 provided with a metal on the surface is used.

The configuration in which at least part of the organic film on the surface of the particle 10 is covered by the blocking agent exemplified by the ethanolamine 40 has been described. However, the blocking agent need not be provided, and a blocking agent other than the ethanolamine 40 may be used.

The configuration in which the SAM 12 is used as the organic film has been described. However, an organic film other than the SAM 12 may be used.

The base material 11 may include the fluorophore F and the paramagnetic material M or may include neither of them. The modified particle 100 may be constructed by using a base material 11 optionally containing a substance having a property suitable for the detection method. The modified particle 100 may contain a coloring agent and, in this case, may also be used as a labeled particle in immunochromatography.

The modified particle 100 can be applied to the detection apparatus 50 described in the embodiment. However, the detection apparatus for the analyte 59 by using the modified particle 100 is not limited to this. For example, in the detection apparatus, a dielectric material such as polystyrene may be used as the base material of the modified particle 100, and only modified particles that have bound to the analyte 59 may be separated and detected by using a dielectrophoresis method.

For example, application to a flow cytometer in which the modified particles 100 are passed one by one through a detection flow passage by using a laminar flow may be performed. For example, a green fluorescent protein is divided into parts such that a substance resulting from association of the divided parts realizes fluorescence, the divided parts are bound to two modified particles, respectively, and a system in which the fluorescence is realized only when both modified particles are bound to the analyte 59 is constructed. A detection apparatus may be constructed by combining such a system with a spectrophotometer. A detection apparatus including the above-described optical system as a detection array capable of performing processing with high throughput may also be realized,

In the above-described embodiment and example, the particle 10 is not a cell, but the particle 10 is not limited to this. If a new particle is realized in accordance with progress of technology or derivation of another technology, the particle may be used to form the modified particle 100, as a matter of course. For example, it is possible that biotechnology is applied, and in this case, the particle 10 may be a cell or the like.

The present disclosure has high storage stability in a liquid and is useful from the viewpoint of applicability to biosensors and the like for research, medical care, and environmental measurement. The modified particle according to the present disclosure and detection apparatuses using the modified particle can be applied to not only non-competitive immunoassay (sandwich immunoassay) but also competitive immunoassay and a gene detection method based on hybridization.

What is claimed is:

1. A modified particle comprising:
a particle;
a specific binding substance that has a property of specifically binding to an analyte and that has been fixed to a surface of the particle; and
an amino sugar molecule fixed to the surface of the particle with an amide bond.

2. The modified particle according to claim 1,
wherein
the particle includes a base material and an organic film covering at least part of a surface of the base material, and
the amino sugar molecule has been fixed to the organic film with the amide bond.

3. The modified particle according to claim 2, further comprising:
a blocking agent that covers at least part of the organic film and that has a property of inhibiting interaction between the organic film and a predetermined molecule.

4. The modified particle according to claim 2,
wherein the organic film is a self-assembled monolayer.

5. The modified particle according to claim 2,
wherein the base material contains a fluorophore.

6. The modified particle according to claim 2,
wherein the base material contains a paramagnetic material or a dielectric material.

7. A method for manufacturing modified particles, comprising:
preparing particles;
fixing a specific binding substance that has a property of specifically binding to an analyte to surfaces of the particles; and
fixing an amino sugar molecule to the surfaces of he particles by using an amide bond.

8. The method for manufacturing modified particles according to claim 7,
wherein the fixing of the specific binding substance and the fixing of the amino sugar molecule are performed by mixing a solution containing the specific binding substance and the amino sugar molecule with the particles.

9. A detection apparatus comprising:
a container for storing the modified particle according to claim 1;
an introducer for introducing, to the container, a sample that may contain an analyte to which the modified particle specifically binds; and
a detector for outputting a detection signal based on an amount of the analyte to which the modified particle has bound.
